(19) United States
(12) Patent Application Publication
(10) Pub. No.: US 2009/0281602 A1
(43) Pub. Date: Nov. 12, 2009

(54) Static electric therapy apparatus

(75) Inventors: Koji Hirasawa, Yokohama-shi (JP); Osamu Ide, Funabashi-shi (JP); Shigeaki Yoshikawa, Nankoku-shi (JP); Takashi Tachibana, Kochi-shi (JP); Ryo Tamura, Kochi-shi (JP)

Correspondence Address: BIRCH STEWART KOLASCH & BIRCH, PO BOX 747, FALLS CHURCH, VA, 22040-0747, US

(73) Assignees: NATURALLY YOURS CO., LTD.; NIHON TRIM CO., LTD.

(21) Appl. No.: 12/149,814

(22) Filed: May 8, 2008

(51) Int. Cl. A61N 1/20; A61N 1/04

(52) U.S. Cl. 607/63; 607/148

(57) ABSTRACT

A static electric therapy apparatus is disclosed which does not leave a user discomfort. The static electric therapy apparatus comprises an electric potential generator connected to an alternating power supply, and an output terminal which provides a human body with electric potential by touching the human body, which output terminal is connected to the electric potential generator; and the static electric therapy apparatus is characterized in that it further comprises a ground wire connected to the output terminal; a direct-current power supply applying negative voltage to the output terminal, which is connected to the output terminal in series; and a transfer switch which switches the case where the output terminal is connected to the potential generator and the case where the output terminal is connected to the ground wire, the switch being connected to the output terminal. Also, the static electric therapy apparatus comprises an electric potential generator connected to an alternating power supply, and an output terminal which provides a human body with electric potential by touching the human body, which output terminal is connected to the electric potential generator, and the static electric therapy apparatus is characterized in that it farther comprises a voltage reducer which gradually decreases the voltage applied to the output terminal when finishing the use of the static electric therapy apparatus.

### FIELD OF THE INVENTION

The present invention relates to a static electric therapy apparatus,

### DESCRIPTION OF THE RELATED ART

A static electric therapy apparatus displays therapeutic effect on headache, stiff shoulders, chronic constipation and the like by applying a voltage of 1000 V to several thousand V to a human body, and static electric therapy apparatuses which employ an alternating current 100 V power supply for household use are also commercially available. Mat-type static electric therapy apparatuses in which an electrode in the form of a mat is incorporated are also commercially available, on which mat a user lies and a high voltage is applied. Alternatively, the mat-type static electric therapy apparatus is used while the mat is laid on which only the feet of the user are placed (Patent Literatures 1 and 2).

On the other hand, a co-inventor of the present application previously invented an apparatus for assisting relaxation which brings relaxation effect by applying a negative direct current voltage to a human body with the body grounded (Patent Literature 3).

Patent Literature 1: JP 2005-21465

Patent Literature 2: JP 2000-42123

Patent Literature 3: WO02/28464

### SUMMARY OF THE INVENTION

It is known mat there is a case where the conventional static electric therapy apparatus leaves a discomfort like a bathing reaction after the voltage is disconnected after the usage.

An object of the present invention is to provide a static electric therapy apparatus which does not leave the discomfort after a static electric therapy.

The present inventors intensively studied to find that the problem of the discomfort after the static electric therapy can be avoided by grounding the human body while applying a negative direct current voltage to the human body, and to reach the idea that if the static electric therapy apparatus is so constituted as to be capable of grounding while applying a negative direct current voltage to the output terminal by switching a switch, not only the user may receive a grounding process without changing the posture after the static electric therapy so that the apparatus gives enhanced convenience, but also the complication and upsizing of the apparatus are minimized, thereby completing the present invention.

Further, the inventors intensively studied to find that the main reason for the discomfort like a bath reaction after disconnecting the voltage is due to instantaneous disconnection of the voltage which is applied to the human body and that the discomfort is greatly improved by not instantaneously disconnecting the high voltage but gradually decreasing the voltage, thereby completing the present invention.

That is, the present invention provides a static electric therapy apparatus comprising an electric potential generator connected to an alternating power supply; and an output terminal which provides a human body with electric potential by touching the human body, the output terminal being connected to the electric potential generator; characterized in that the static electric therapy apparatus further comprises a ground wire connected to the output terminal; a direct-current power supply applying negative voltage to the output terminal, which is connected to the output terminal in series; and a transfer switch which switches the case where the output terminal is connected to the potential generator and the case where the output terminal is connected to the ground wire, the switch being connected to the output terminal.

The the present invention also provides a static electric therapy apparatus comprising an electric potential generator connected to an alternating power supply; and an output terminal which provides a human body with electric potential by touching the human body, the output terminal being connected to the electric potential generator; characterized in that the static electric therapy apparatus further comprises a voltage reducer which gradually decreases the voltage applied to the output terminal when finishing the use of the static electric therapy apparatus.

### EFFECTS OF THE INVENTION

By the present invention, a static electric therapy apparatus by which the user may receive a process preventing the discomfort after the static electric therapy without changing the posture of the user was first provided. It is highly convenient for the user to use the static electric apparatus of the present invention because the user may receive, in the same posture as the one during the static electric therapy, a process for preventing Hie discomfort. Further, since the output terminal of the static electric apparatus is used as it is as a ground wire for the process for preventing the discomfort, the complication and upsizing of the apparatus are minimized.

Further, by the present invention, a static electric therapy apparatus by which the discomfort like a bath reaction which is left after the static electric therapy is alleviated was first provided.

### BRIEF DESCRIPTION OF THE DRAWINGS

FIG. 1 is a circuit diagram illustrating one embodiment of the static electric therapy apparatus of the first invention of the present application.

FIG. 2 is a block diagram illustrating one embodiment of the static electric therapy apparatus of the second invention of the present application.

FIG. 3 is a block diagram illustrating another embodiment of the static electric therapy apparatus of the second invention of the present application.

### BEST MODE FOR CARRYING OUT THE INVENTION

As mentioned above, the static electric therapy apparatus of the first invention of the present application is based on the known static electric therapy apparatus comprising an electric potential generator connected to an alternating power supply and an output terminal supplying an electric potential to a human body by touching the human body, which output terminal is connected to the electric potential generator, and is characterized by further comprising a ground wire connected to the output terminal; a direct-current power supply applying negative voltage to the output terminal, which power supply is connected to the output terminal in series; and a transfer switch which switches the case where the output terminal is connected to the electric potential generator and the case where the output terminal is connected to the ground wire, which switch is connected to the output terminal.

It is preferred that the ground wire be connected to the output terminal through the direct current power supply. It is also preferred that the ground wire be connected to the ground side of an alternating power supply for household use because the more enhanced convenience is obtained. That is, one terminal of the alternating power supply for household use is a ground wire, and it is preferred that the output terminal be grounded by connecting the output terminal of the static electric therapy apparatus to the ground side of the alternating power supply. Which of the two terminals of the alternating power supply is the ground is easily detected by incorporating a well-known ground detection circuit (for example, described in Patent Literature 3). The connection to the ground terminal may be performed manually by a transfer switch or performed automatically by interlocking the ground detection circuit with the transfer switch. It is preferred mat a resistor be located between the direct current power supply and the output terminal in order to prevent an electric shock. It is suitable to use a resistor having a resistance of 100 ohms to 1 k ohms. It is possible to use a variable resistor having a resistance of about 1 k ohms and to decrease the value of resistance gradually from high to low.

Although the negative voltage is conveniently applied by using a battery, a direct current voltage can be generated from a power source for household use by using a converter. From the viewpoint of preventing the discomfort after the static electric therapy, it is preferable to use a negative voltage of −12 V to −27 V, more preferably −14 V to −22 V.

The switch mat switches from the static electric therapy to the grounding and negative voltage applying process (hereinafter referred to as “grounding process” for convenience) may be a manual switch, or may be automatically switched to the grounding process after a predetermined time has passed by being interlocked with a timer.

The other constitution may be the same as the conventional static electric therapy apparatuses except that the grounding process means is provided in which the output terminal of the static electric therapy apparatus is grounded as described above. The apparatus which employs an alternating current power supply for household use as a power supply is preferred because it has enhanced convenience. For the output terminal, a mesh sheet which is formed of metal fiber or plastic fiber, or a mat which incorporates the mesh sheet or the like is preferably employed. The voltage is normally from 1000 V to several thousand V. A constitution in which a timer is incorporated and the voltage is gradually decreased to zero V over several minutes to ten and several minutes is preferred.

In operation, in the case of using an output terminal in the form of a mat, a static electric therapy is firstly performed in a normal way, for example, by lying on the terminal or placing the user's feet on the terminal. After the electric therapy, preferably after the output terminal is switched to the grounding process side after the voltage is gradually decreased to zero, the output terminal is grounded and, simultaneously, a negative direct current voltage is applied to the output terminal. This switching process may be performed by using a timer to enable the automatic switching as described above. The state in which the negative direct current voltage is applied to the human body and, simultaneously, the human body is grounded is maintained preferably for several minutes. Since the state in which the negative direct current voltage is applied to the human body while the human body is grounded does not adversely affect the human body, rather will provide a relaxation effect to the human body, the user may sleep after the therapy in the state where the switch is set to the grounding process. In this way, by performing the grounding process after the static electric therapy, the discomfort which may be left after the static electric therapy using a high voltage may be effectively prevented.

The first invention of the present application will now be described more concretely by way of an embodiment thereof. However, the first invention of the present application is not restricted to the following embodiment.

Based on FIG. 1, a preferred embodiment of the first invention of the present application will be described. In FIG. 1, reference numeral 10 denotes a potential generator, and it is connected to an alternating current 100 V power supply for household use through power supply cords 12a and 12b. Reference numeral 12b denotes the ground side of the alternating current 100 V power supply for household use. A potential generator 10 is connected to an output terminal 16 through a terminal 14a of a switch 14. The output terminal in the form of a mesh sheet made of copper wire, enclosed in a bag or the like made of an air-permeable cloth to form a sheet or mat, is preferably used. Reference numeral 17 denotes an operation-switching timer and 18 is a voltage controller. When a predetermined time set in the operation-switching timer 17 has passed, the voltage controller 18 operates, and then the voltage which is generated by the potential generator 10 is gradually decreased to zero V over several minutes to ten and several minutes. As the operation-switching timer 16 and the voltage controller 18, those well-known which are used for commercially available static electric therapy apparatuses may be employed. The constitution described above is the well-known one which is adopted for commercially available static electric therapy apparatuses.

A terminal 14b in the transfer switch 14 is connected to the negative electrode of a battery 22 through a resistor 20. In this embodiment, the resistor 20 has a resistance of 330 ohms, and the battery, which comprises series-connected two 9V dry cells, has a voltage of 18 V. A battery checker circuit not shown may be connected to the battery 22. The battery checker circuit per se is well-known. An example of the battery checker circuits is the one which turns on an alarm lamp when the voltage of the battery decreased below the predetermined value of, e.g., 14 V by using a Zener diode, or the like. The positive electrode of the battery 22 is connected to an alternating current 100 V power supply for household use through a ground detection apparatus and a transfer switch (hereinafter referred to as “ground transfer switch” for convenience) 24. A terminal 24b in the ground transfer switch 24 is connected to the ground side 12b of the alternating current 100 V power supply for household use, and the other terminal 24a is connected to the non-ground side 12a. The ground detection circuit per se is well-known. The circuit detects whether the transfer switch 24 is connected to the ground side 12b or not and if the switch is not connected to the ground side, the circuit connects the positive electrode of the battery 22 to the ground side 12b of the alternating current 100 V power supply for household use by switching the switch. A switch which automatically detects the ground side and switches to the ground side automatically may be employed as the ground transfer switch 24.

In operation, a user firstly receives a static electric therapy in the same way as a therapy by a normal static electric therapy apparatus. That is, the time to perform the static electric therapy is set beforehand by using the timer 17. The transfer switch 14 is connected to the terminal 14a. Then, in the case of using a static electric therapy apparatus where the output terminal 16 is a mesh made of copper wire, the mesh being stored in an air-permeable cloth bag which together makes a sheet, the user lays on the sheet or the user's feet are placed on the sheet. In this state, the power is turned on, a high voltage generated by the electric potential generator 10 is applied to the human body through the output terminal 16. When the time set by the timer passes, the timer works and the voltage controller 18 tied to the timer works whereby the voltage generated by the potential generator 10 is gradually decreased. When the voltage is decreased to zero over several minutes to ten and several minutes, the transfer switch 14 is switched to the terminal 14b. On the other hand, the ground side is detected by the ground transfer switch 24 and then the ground transfer switch 24 is switched to the terminal 24b (if the ground transfer switch is connected to the ground side, there is naturally no need to switch of course). In this case, the human body is grounded with a negative direct current voltage of 18 V applied. The discomfort due to the static electric therapy may thus be prevented. The grounding process is preferred to be performed at least for several minutes. The grounding process will not cause side effects such as discomforts at all, rather the state where the human body is grounded is preferred, and therefore the user may sleep overnight in the state where the user receives the grounding process.

On the other hand, the static electric therapy apparatus of the second invention of the present application is, as described above, a static electric therapy apparatus comprising an electric potential generator connected to an alternating power supply; and an output terminal which provides a human body with electric potential by touching the human body, the output terminal being connected to the electric potential generator; characterized in that the static electric therapy apparatus further comprises a voltage reducer which gradually decreases the voltage applied to the output terminal when finishing the use of the static electric therapy apparatus.

As described above, static electric therapy apparatuses apply a voltage of about 800 V to several thousand V to a human body. As for the static electric therapy apparatus of the present invention, when finishing the use of the apparatus, the high voltage which is applied to the human body is gradually decreased, normally over several minutes to several tens minutes, preferably over several minutes to ten and several minutes, and finally the power is turned off (the voltage is 0 V.). The gradual decrease in the voltage may be a continuous one or a stepwise one. When the voltage decrease is a stepwise one, the voltage is decreased stepwise, normally in steps of about 50 V to 500 V, preferably in steps of about 100 V to 300 V. All the steps of the voltage decrease is not necessarily the same, rather it is preferable that the lower the voltage decreases, the smaller the step of the voltage decreased becomes.

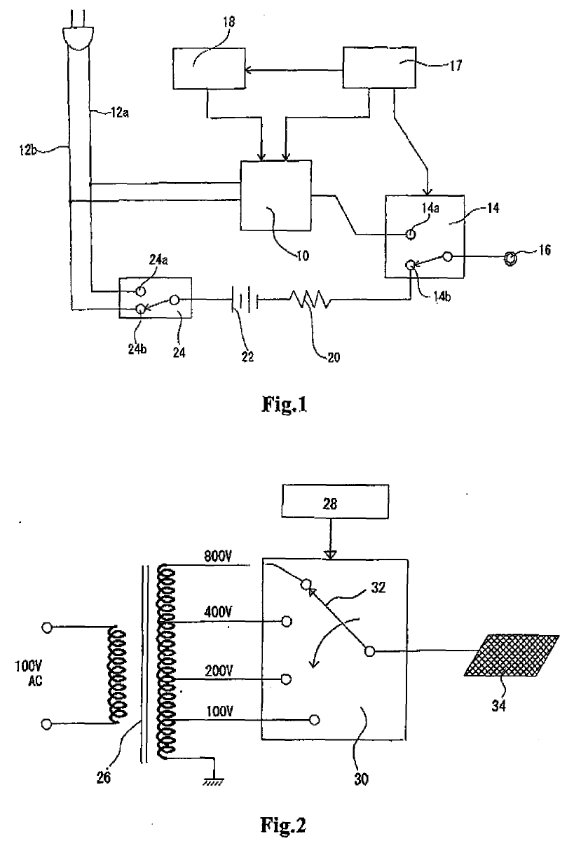

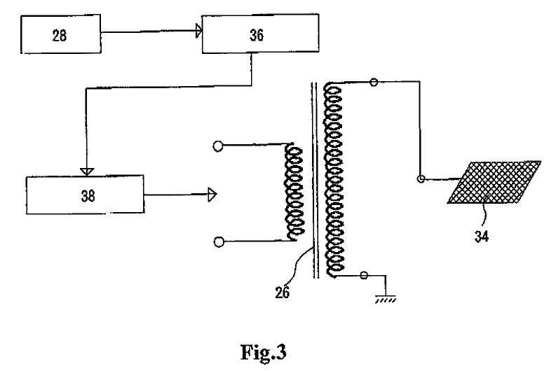

Means for gradually decreasing the voltage applied to the output terminal (hereinafter also referred to as “voltage gradually-decreasing means”) is per se obvious for those skilled in the art, and various means arc possible. For example, those comprising a high voltage transformer which is said potential generator which generates a plurality of different voltages and which is equipped with a plurality of terminals; and a relay-operated tap changing circuit interlocked with a timer and which switches the terminal to be connected from a higher voltage generating terminal to a lower voltage generating terminal successively in said plurality of terminals may be exemplified. This is schematically illustrated in FIG. 2. In FIG. 2, reference numeral 26 denotes a high voltage transformer which is a potential generator, 28 denotes a timer, 30 denotes a relay-operated tap changing circuit, 32 denotes a rotary switch, and 34 denotes an output terminal in the form of a sheet The relay-operated tap changing circuit interlocked with a timer is per se well-known. The high voltage transformer 26 comprises a plurality of terminals on which a plurality of different voltages are generated (four kinds of terminals indicated by 800 V, 400 V, 200 V and 100 V in the figure), the rotary switch is controlled by a timer, and the switch rotates when a predetermined time has elapsed, thus the terminal to be connected is switched to the adjacent terminal (in the embodiment shown in FIG. 2, the switch rotates counterclockwise as illustrated by an arrow in FIG. 2). The voltage is thus decreased stepwise. Finally, the power is turned off by the timer 28, and the voltage reaches 0 V.

Another embodiment of the voltage gradually-decreasing means is shown in FIG. 3. In the example shown in FIG. 3, the voltage gradually-decreasing means is constituted by a timer 28, a voltage control circuit 36 connected to the timer, a variable output inverter 38 which changes the generated voltage from a high voltage transformer 26 that is the potential generator which inverter is connected to the voltage control circuit 36. The voltage control circuit 36 and the variable output inverter 38 per se are well-known.

The other constitution may be the same as the conventional static electric therapy apparatuses except that the voltage gradually-decreasing means is provided as described above. The apparatus which employs an alternating current power supply for household use as a power supply is preferred because it has enhanced convenience. For the output terminal, a mesh sheet which is formed of metal fiber or plastic fiber, or a mat which incorporates the mesh sheet is preferably employed The voltage is normally from 800 V to several thousand V.

In operation, in the case of using an output terminal having the form of a mat, a static electric therapy is firstly performed in a normal way, for example, by lying on the terminal or placing the user's feet on the terminal. After the electric therapy, the voltage is gradually decreased by the voltage gradually-decreasing means before the power is turned off (the voltage reaches 0 V). In this way, by gradually decreasing the voltage, the discomfort which is suspected to be left after the static electric therapy using a high voltage may be effectively prevented.

1. A static electric therapy apparatus comprising:
an electric potential generator connected to an alternating power supply; and
an output terminal which provides a human body with electric potential by touching the human body, said output terminal being connected to said electric potential generator; characterized in that said static electric therapy apparatus further comprises:
a ground wire connected to said output terminal;
a direct-current power supply applying negative voltage to said output terminal, which is connected to said output terminal in series; and
a transfer switch which switches the case where said output terminal is connected to said potential generator and the case where said output terminal is connected to said ground wire, said switch being connected to said output terminal.

2. The static electric therapy apparatus according to claim 1, wherein said ground wire is connected to said output terminal via said direct current power supply.

3. The static electric therapy apparatus according to claim 1 or 2, wherein said ground wire is connected to the ground side of an alternating current power supply for household use.

4. The static electric therapy apparatus according to claim 1, wherein a resistor is located between said direct current power supply and said output terminal.

5. The static electric therapy apparatus according to claim 1, wherein said output terminal is in the form of a mesh sheet.

6. A static electric therapy apparatus comprising:
an electric potential generator connected to an alternating power supply; and an output terminal which provides a human body with electric potential by touching the human body, said output terminal being connected to said electric potential generator; characterized in that said static electric therapy apparatus further comprises:
a voltage reducer which gradually decreases the voltage applied to said output terminal when finishing the use of said static electric therapy apparatus.

7. The static electric therapy apparatus according to claim 6, wherein said voltage reducer comprises a high voltage transformer which is said potential generator which generates a plurality of different voltages and which is equipped with a plurality of terminals; and a relay-operated tap changing circuit interlocked with a timer and which switches the terminal to be connected from a higher voltage generating terminal to a lower voltage generating terminal successively in said plurality of terminals.

8. The static electric therapy apparatus according to claim 6, wherein said means for gradually decreasing the voltage comprises a timer; a voltage control circuit connected to said timer; a variable output inverter that changes the generated voltage from a high voltage transformer which is said potential generator; said variable output inverter being connected to said voltage control circuit.

9. The static electric therapy apparatus according to any one of claims 6 to 8, wherein said output terminal is in the form of a mesh sheet.
